(19) United States
(12) Patent Application Publication
(10) Pub. No.: US 2023/0002743 A1
(43) Pub. Date: Jan. 5, 2023

(54) SELF-ASSEMBLED CATALASE NANOPARTICLE AND PREPARATION METHOD THEREFOR AND USE THEREOF

(71) Applicant: ZHEJIANG GONGSHANG UNIVERSITY, Hangzhou, Zhejiang (CN)

(72) Inventors: PINGFAN RAO, Hangzhou, Zhejiang (CN); LIJING KE, Hangzhou, Zhejiang (CN); JIANWU ZHOU, Hangzhou, Zhejiang (CN); ZHAOSHUO YU, Hangzhou, Zhejiang (CN); SIHAO LUO, Hangzhou, Zhejiang (CN); GUANZHEN GAO, Hangzhou, Zhejiang (CN); HUIQIN WANG, Hangzhou, Zhejiang (CN)

(21) Appl. No.: 17/623,166

(22) PCT Filed: Apr. 16, 2020
(86) PCT No.: PCT/CN2020/085054

§ 371 Date: Jan. 28, 2022

(30) Foreign Application Priority Data
Jun. 24, 2019 (CN) 201910549698.5

(51) Int. Cl. C12N 9/08; A61K 38/44; A61K 9/51; B01D 61/14; A23L 29/00

(52) U.S. Cl. CPC C12N 9/0065; C12Y 111/01006; A61K 38/44; A61K 9/51; B01D 61/147; A23L 29/06; A23V 2002/00

(57) ABSTRACT

Disclosed are a self-assembled catalase nanoparticle and a preparation method therefor and the use thereof. The self-assembled catalase nanoparticle of the present invention is obtained by dissolving catalase freeze-dried powder to obtain a catalase solution, adjusting the pH value of the catalase solution, and then centrifugating or filtering same to obtain a supernatant or a filtrate, and further thermally incubating the supernatant or filtrate. The self-assembling catalase nanoparticle of the present invention can be used in medicines or food products that promote immune cell growth and regulate organic immunity.

### TECHNICAL FIELD

The invention belongs to the technical field of biology, food and nano materials, and particularly relates to a self-assembled catalase nanoparticle as well as a preparation method therefor and application thereof.

### BACKGROUND

Catalase (CAT) is an enzyme that catalyzes the decomposition of hydrogen peroxide into oxygen and water, is present in the peroxide body of cells, is an important component of redox balance in the body, and can eliminate free radicals such as excessive hydrogen peroxide in the body to form an important barrier for the body immunity. In recent years, exogenous catalase has been widely used for immune-related therapy for tumor diseases, and further development of biomaterials based on catalase having higher physiological activity is urgently needed. At present, researchers at home and abroad have developed a plurality of catalase simulation drugs capable of scavenging free radicals, but the preparation routes of the mimics are complex, and the safety in vivo is not clear.

Currently, nanotechnology is receiving much attention in many fields, because it has great advantages brought by nanoscale, such as greatly enhancing the original functional properties of active ingredients at molecular scale, being more accessible to cells, good targeting, high efficiency of action, etc. Based on this, many researches focus on the research and development of catalase nanoparticles to obtain nano biomaterials with more remarkable physiological performance, such as a shearing force combined organic reagent method, a calcium carbonate wrapping method and the like, so that the stability of catalase is improved, however, the methods have the problems of residual chemical reagents, high safety risk, complex and uncontrollable preparation and the like.

At present, no report related to the preparation and research of catalase nanoparticles formed by self-assembly exists at home and abroad. The invention utilizes the principle that the hydrophobic interaction between proteins is increased under the heat treatment condition, and larger-scale supramolecular polymers are easier to form, uses catalase from bovine liver as a raw material, adopts a thermal processing incubation mode, controls the temperature and the pH value, and prepares active catalase nanoparticles in a green way.

### SUMMARY OF THE INVENTION

The invention aims to provide a self-assembled catalase nanoparticle and a preparation method therefor and uses thereof. The preparation method disclosed by the invention is green and safe, is simple to operate, and the obtained self-assembled catalase nanoparticles are controllable in particle size and have higher activity and stability.

In order to achieve the purpose, the invention adopts the following technical scheme:

a method for preparing self-assembled catalase nanoparticles, comprising the following steps: dissolving catalase freeze-dried powder to obtain a catalase solution, adjusting the pH value of the catalase solution, centrifuging or filtering to obtain a supernatant or a filtrate, and further performing thermal incubation on the supernatant or the filtrate to obtain the catalase nanoparticles.

Further, in the preparation method, the concentration of the catalase solution is 0.2-5 mg/mL; preferably, the concentration of the catalase solution is 0.2-1.6 mg/mL.

Further, in the preparation method, the pH value of the catalase solution is adjusted to 6.5-9.5; preferably, the pH value is 7.0-8.0.

Further, in the preparation method, the centrifugation speed is 2000-5000 rpm/min.

Further, in the preparation method, the filtration is membrane filtration, and the pore size of the membrane filtration is 0.22-0.45 μm.

Further, in the preparation method, the heat incubation is to heat the supernatant or the filtrate at the temperature of 45-90° C. for 15 s-15 min; the higher the heating temperature of the invention, the shorter the heating time required. For example, only 15-60 seconds of heating is required at a temperature of 65-90° C.

The invention also provides the self-assembled catalase nanoparticles obtained by the preparation method, wherein the particle size of the self-assembled catalase nanoparticles is 30-200 nm.

Further, the enzyme activity of the self-assembled catalase nanoparticles is more than or equal to 3100 U/mg.

The invention also provides an application of the self-assembled catalase nanoparticle in food or medicines.

Further, the self-assembled catalase nanoparticle is applied to food or medicine with an immune regulation function.

The principle of the invention is as follows:

the invention removes overlarge polymer impurities in the catalase freeze-dried powder by using a centrifugal and filtering method, and then under a heat treatment condition, the hydrophobic effect between catalase proteins is increased, so that a larger-scale supermolecule polymer is easily formed, and self-assembled catalase nanoparticles with larger nanometer sizes are formed by self-assembly.

The invention has the following technical characteristics:

(1) the self-assembled catalase nanoparticles provided by the invention are green and safe to prepare, do not need chemical crosslinking and structure, and are simple to operate.
(2) The self-assembled catalase nanoparticles related to the invention can be used for accurately preparing the self-assembled catalase nanoparticles with different particle sizes by adjusting the pH value of the system, so that different physiological effects are generated, and the obtained self-assembled catalase nanoparticles have centralized and uniform particle size distribution.
(3) Compared with the original natural catalase, the self-assembled catalase nanoparticle provided by the invention has greatly improved stability.

### BRIEF DESCRIPTION OF THE DRAWINGS

FIG. 1 effect of different pH on self-assembled catalase nanoparticle formation: a) average particle size plot; b) zeta-potential map.

FIG. 2 particle size distribution of self-assembled catalase nanoparticles: a), b), c) are catalase nanoparticles formed under pH 7.0, 7.2, 8.0, respectively.

FIG. 3 SDS-PAGE electrophoresis of self-assembled catalase nanoparticles: a) non-reduced electrophoresis, from left to right, of solubilized native CAT and CAT nanoparticles (pH 7.0, 7.2, 7.4, 7.6, 7.8, 8.0) at different pH conditions (pH 7.0, 7.2, 7.4, 7.6, 7.8, 8.0); b) reduced electrophoresis (sequence as above).

FIG. 4 SEM electron micrograph (a) and round dichroism (b) of self-assembled catalase nanoparticles.

FIG. 5 enzyme activity of self-assembled catalase nanoparticles prepared at different pH.

FIG. 6 effect of self-assembled catalase nanoparticles prepared at different pH on macrophage survival.

### DETAILED DESCRIPTION OF THE INVENTION

In order to make the objects, technical solutions and advantages of the embodiments of the present invention clearer, the technical solutions of the embodiments of the present invention will be clearly and completely described below. It is to be understood that the embodiments described are only a few embodiments of the present invention, and not all embodiments. All other embodiments, which can be derived by a person skilled in the art from the described embodiments of the invention without any inventive step, are within the scope of protection of the invention.

Unless defined otherwise, technical or scientific terms used herein shall have the ordinary meaning as understood by one of ordinary skill in the art to which this invention belongs.

### Example 1

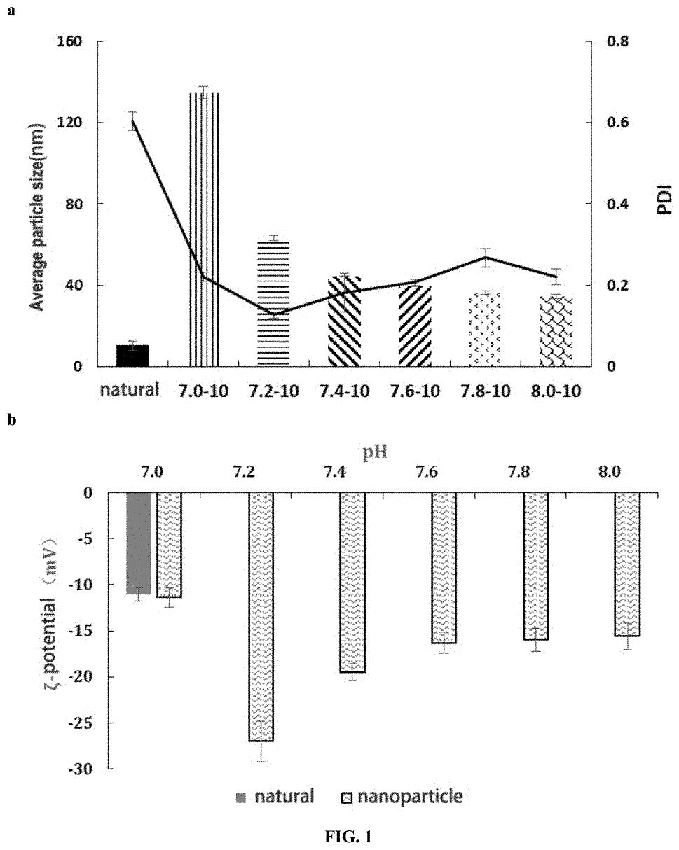

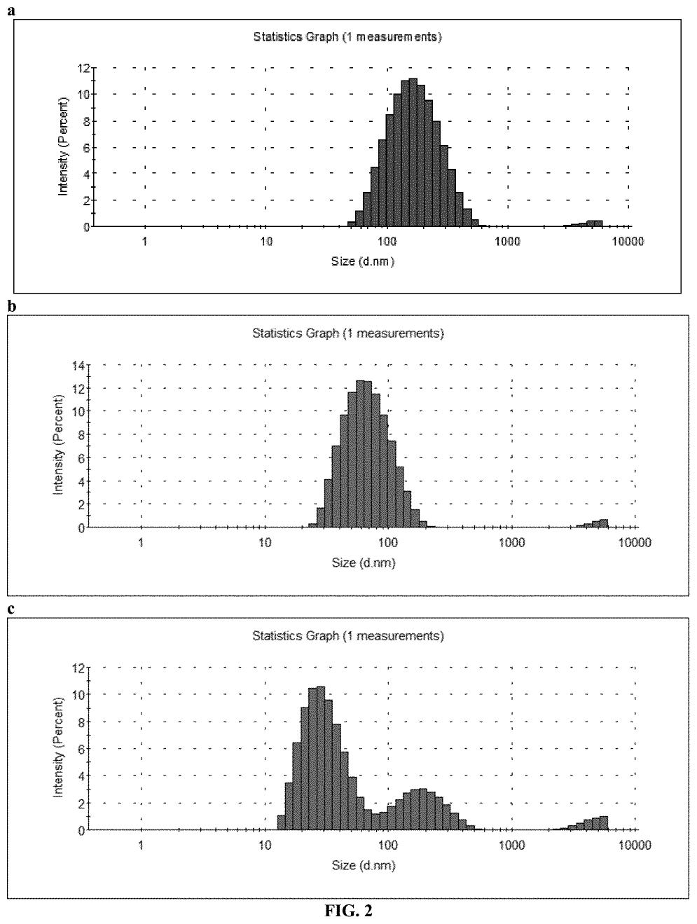

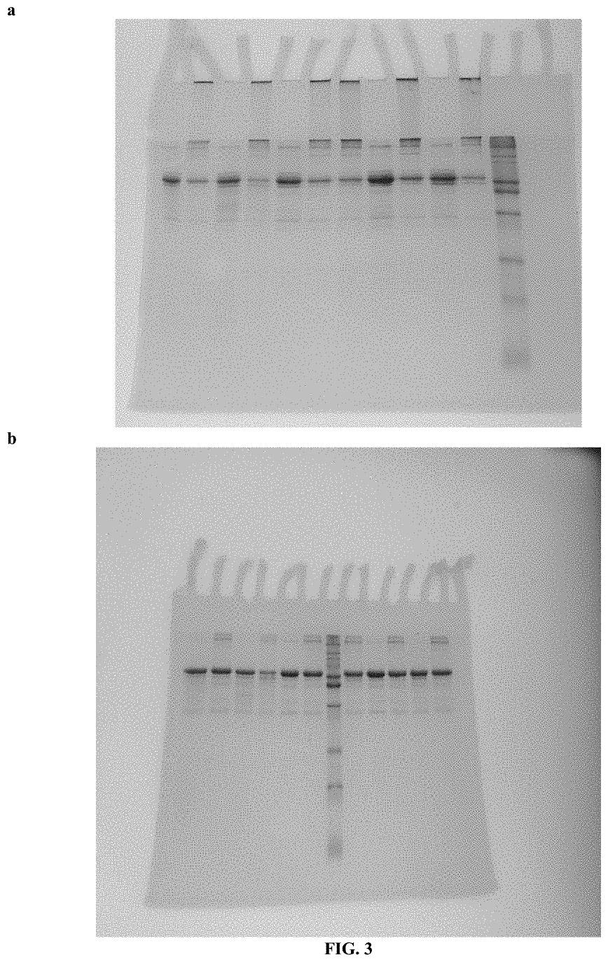

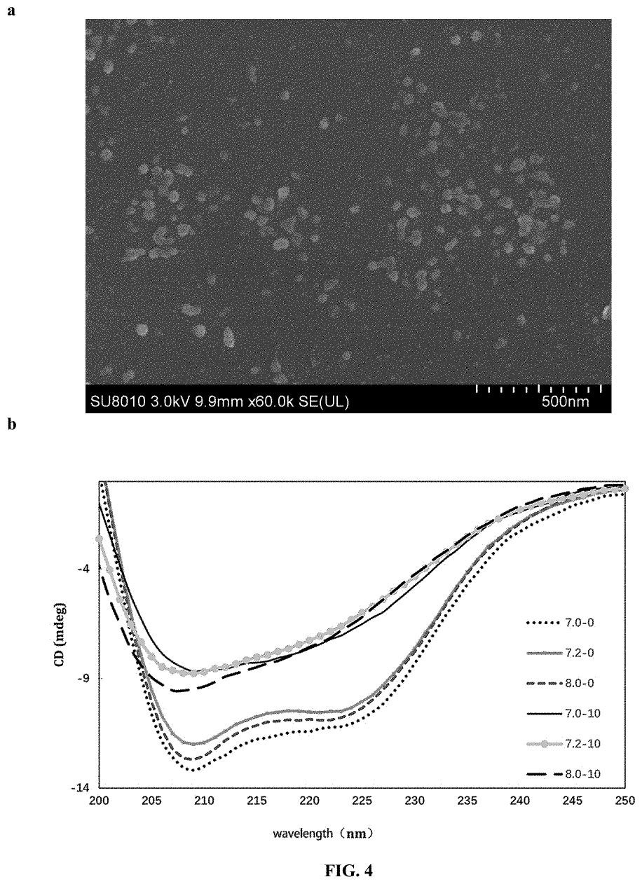

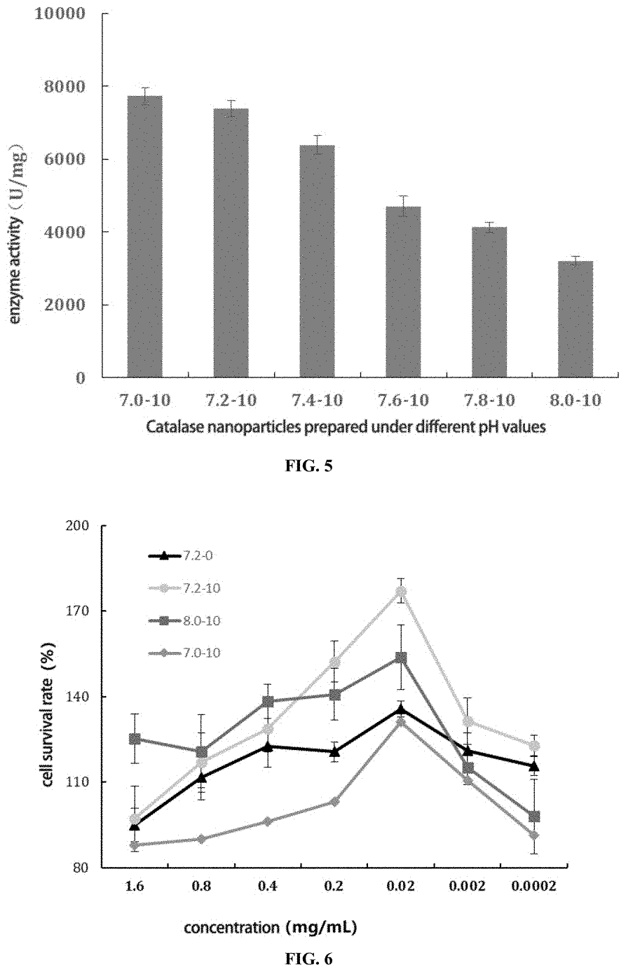

0.8 mg of catalase freeze-dried powder was weighted and dissolved in 1 mL of phosphate buffer solution with pH 7.0 and 0.02M with oscillating to be fully dissolved, then centrifuged at the centrifugal speed of 5000 rpm/min for 10 min, the supernatant was selected, and placed in a 1.5 mL centrifuge tube. And then the centrifuge tube was put into a dry heat incubator, and was incubated for 10 min at the temperature of 60° C. to obtain the self-assembled catalase nanoparticles. The particle diameter and surface potential were measured with laser particle analyzer, wherein the measured particle diameter was 134.90+−3.18 nm, the surface charge range was −11.40+−1.07 mV, as shown in FIG. 1, and the natural in FIG. 1 refers to untreated catalase; the self-assembled catalase nanoparticle size distribution was shown in FIG. 2; the SDS-PAGE electrophoresis was shown in FIG. 3; SEM electron micrograph and CD spectrogram were shown in FIG. 4, and the enzyme activity of the self-assembled catalase nanoparticles was 7732.50+/−235.74 U/mg, as shown in FIG. 5.

### Example 2

0.8 mg catalase freeze-dried powder was weighed, 1 mL of phosphate buffer solution was dissolved with pH 7.2 and 0.02M with oscillating to be fully dissolved, then centrifuged at the centrifugal speed of 5000 rpm/min for 10 min, the supernatant was selected, and placed in a 1.5 mL centrifuge tube. And then the centrifuge tube was put into a dry heat incubator, and was incubated for 10 min at the temperature of 60° C. to obtain the self-assembled catalase nanoparticles. The particle diameter and surface potential were measured with laser particle analyzer to obtain particle diameter of 63.40+−1.23 nm and surface charge range of −27.0+−2.21 mV (see FIG. 1); the self-assembled catalase nanoparticle particle size distribution was shown in FIG. 2; the self-assembled catalase nanoparticle particle size distribution was shown in FIG. 2; the SDS-PAGE electrophoresis was shown in FIG. 3; SEM electron micrograph and CD spectrogram were shown in FIG. 4, and the enzyme activity of the self-assembled catalase nanoparticles was 7382.50+/−221.77 U/mg, as shown in FIG. 5.

### Example 3

0.8 mg of catalase freeze-dried powder was weighed and dissolved in 1 mL of phosphate buffer solution with pH (7.4-8.0) and 0.02M with oscillating to be fully dissolved, then the solution was centrifuged for 10 min at the centrifugal speed of 5000 rpm/min, the supernatant was selected, and the supernatant was placed into a 1.5 mL centrifuge tube. And then the centrifuge tube was placed into a dry heat incubator, and was incubated for 10 min at the temperature of 60° C. to obtain the self-assembled catalase nanoparticles. The particle diameter and surface potential were measured with a laser particle size analyzer to obtain a particle diameter of about 40 nm and a surface charge of −15.6 mV to −19.5 mV (see FIG. 1); the self-assembled catalase nanoparticle size distribution was shown in FIG. 2; the SDS-PAGE electrophoresis was shown in FIG. 3; SEM electron micrograph and CD spectrogram were shown in FIG. 4, and enzyme activity of the nanoparticles was more than or equal to 3100 U/mg and was shown in FIG. 5.

As could be seen from examples 1-3, the particle size of the self-assembled catalase nanoparticles gradually decreased with the increase of pH, so that the size of the self-assembled catalase nanoparticles could be adjusted by controlling the pH value of the system, and the self-assembled catalase nanoparticles could be applied according to different occasions. In addition, in the aspect of stability, after catalase was self-assembled to form a nano size, the absolute value of the zeta-potential standard of storage stability was obviously improved, and meanwhile, the self-assembled catalase nanoparticles could resist SDS hydrolysis, so that the chemical stability was improved.

### Example 4

0.8 mg of catalase freeze-dried powder was weighed and dissolved in 1 mL phosphate buffer solution with pH 7.2 and 0.02M with shaking to fully dissolve, filtering with a membrane with pore diameter of 0.45 μM or 0.22 μM to obtain filtrate, and placed into a 1.5 mL centrifuge tube. And then the centrifuge tube was put into a dry heat incubator and was incubated for 30 s at 75° C. to obtain the self-assembled catalase nanoparticles, the particle size and the surface potential of the nanoparticles were measured by using a laser particle sizer, wherein the measured particle size is about 110 nm, and the enzyme activity of the nanoparticles was more than or equal to 3300 U/mg.

### Example 5

Application on macrophage of the alimentary canal mucous membrane.

The macrophage of the digestive tract mucosa of SD rat was taken as a model, the survival rate was taken as an index, the influence of the self-assembled catalase nanoparticles formed under different pH (pH 7.0, pH 7.2 and pH 8.0) conditions and the influence of natural catalase on the macrophage under different concentrations were discussed, and the biochemical analysis result was shown in FIG. 6.

The results showed that the self-assembled catalase nanoparticles had a stronger ability to promote macrophage growth than native catalase in a certain range, and the highest promotion effect was more than 77%. Therefore, when the nano antioxidant enzyme acts on the body as the nano medicament, not only the enzyme activity of the nano antioxidant enzyme but also the particle size of the formed nano structure is considered so as to maximally exert the physiological activity of the nano antioxidant enzyme.

The above description of the embodiments is only intended to facilitate the understanding of the method of the invention and its core ideas. It should be noted that, for those skilled in the art, it is possible to make various improvements and modifications to the present invention without departing from the principle of the present invention, and those improvements and modifications also fall within the scope of the claims of the present invention.

1. A preparation method of self-assembled catalase nanoparticles, comprising the following steps: dissolving catalase freeze-dried powder to obtain a catalase solution, adjusting the pH value of the catalase solution, then obtaining supernatant or filtrate through centrifugation or filtration, and further performing thermal incubation on the supernatant or the filtrate to obtain the catalase nanoparticles.

2. The method for preparing self-assembled catalase nanoparticles as claimed in claim 1, wherein the concentration of the catalase solution is 0.2-5.0 mg/mL.

3. The method for preparing self-assembled catalase nanoparticles as claimed in claim 1, wherein the pH value of the catalase solution is adjusted to be 6.5-9.5.

4. The method for preparing self-assembled catalase nanoparticles as claimed in claim 1, wherein the centrifugation speed is 2000-5000 rpm/min.

5. The method for preparing self-assembled catalase nanoparticles as claimed in claim 1, wherein the filtration is membrane filtration, and the pore size of the membrane filtration is 0.22 μm-0.45 μm.

6. The method for preparing self-assembled catalase nanoparticles as claimed in claim 1, wherein the heat incubation is performed by heating the supernatant or filtrate at 45-90° C. for 15 s-15 min.

7. The self-assembled catalase nanoparticle obtained by the preparation method of claim 1, wherein the catalase nanoparticle has a particle size of 30 to 200 nm.

8. The self-assembled catalase nanoparticle according to claim 7, wherein the catalase nanoparticle has an enzyme activity of 3100 U/mg or more.

9. Use of the self-assembled catalase nanoparticles of claim 1 in food or pharmaceutical products.

10. Use of the self-assembled catalase nanoparticles in food or pharmaceutical products according to claim 9, wherein the self-assembled catalase nanoparticles are used in food or pharmaceutical products with immunomodulatory functions.
